(19) United States
(12) Patent Application Publication
(10) Pub. No.: US 2001/0039259 A1
(43) Pub. Date: Nov. 8, 2001

(54) Protection of hematopoietic cells by the induction of post-mitotic quiescence

(76) Inventor: Edib Korkut, Bethesda, MD (US)

Correspondence Address: Gary L. Shaffer, Esq., 901 Banks Place, Alexandria, VA, 22312, US

(21) Appl. No.: 09/881,669

(22) Filed: Jun. 18, 2001

Related U.S. Application Data
(63) Continuation of application No. 09/220,677, filed on Dec. 23, 1998.
(60) Provisional application No. 60/096,533, filed on Aug. 13, 1998.

(51) Int. Cl. A61K 38/00

(52) U.S. Cl. 514/2

(57) ABSTRACT

Methods for minimizing the toxic effects of chemotherapy or cytotoxic irradiation on the hematopoietic cells of a patient having neoplastic cells or a malignant tumor are provided. Methods of the invention comprise the steps of treating the patient with a dosage of at least one hematopoietic cell stimulating factor, the dosage being sufficient in amount and time to cause a substantial increase in the population of the hematopoietic cells, and in differentiated blood cells, and then treating the patient with a dosage of chemotherapeutic agents or cytotoxic irradiation sufficient to substantially reduce the population of neoplastic or cancerous cells. The methods increase the absolute number of hematopoietic progenitor cells and differentiated cells in the patient's blood system prior to the administration of therapeutic insult thereby increasing the number of hematopoietic progenitor cells and differentiated cells which survive therapeutic insult.

The present application is related to U.S. Provisional Patent Application No. 60/096,533, filed Aug. 13, 1998.

### BACKGROUND OF THE INVENTION

The present invention pertains to methods for minimizing the toxic effects of chemotherapies on bone marrow, blood cells, and other tissues by the use of factors, which stimulate hematopoietic cells prior to the administration of chemotherapeutic agents.

The use of many known chemotherapeutic drugs to treat cancers and similar disease states is premised on the fact that neoplastic cells, because they are dividing more rapidly than normal cells, are more sensitive than normal cells to the toxic effects of chemotherapeutic drugs. In conventional chemotherapy, neoplastic cells are preferentially killed over normally dividing cells. However, other rapidly dividing but non-neoplastic cells are damaged or killed by the toxic effects of chemotherapy. For example, rapidly dividing yet non-neoplastic cell populations, such as the progenitor cells of the hematopoietic system and oral epithelial cells, both of which normally maintain a high rate of proliferation relative to other non- cancerous cell types, are also sensitive to chemotoxicity. Cytotoxic damage to these sensitive-though-non-neoplastic cells is thus a common and very serious side effect of chemotherapy, a side effect which commonly limits the dosage of chemotherapeutic drugs that can be tolerated by a patient.

It is well known that chemotherapeutic drugs kill a fixed fraction or percentage of rapidly dividing cells. For example, a particular chemotherapeutic regimen for renal cancer might kill 90 percent of the absolute number of cells with 10 percent of the cells surviving. Thus, if 100,000 cells were treated, then 90,000 cells would be killed and 10,000 cells would survive. Similarly, if 10,000,000 cells were treated, then 9,000,000 cells would be killed and 1,000,000 cells would survive. This general principle of chemotherapy holds true for the cytotoxic effects of chemotherapeutic drugs on rapidly dividing yet non-neoplastic cell populations, such as the progenitor cells of the hematopoietic system. A corollary to this general principle is that the absolute number of cells, which survive chemotherapy, is larger when the beginning cell population is larger. Thus, a method, which increases the absolute number of a pre-chemotherapy population of, cells results in an increased absolute number of those cells, which survive the chemotherapy.

Proliferative activity influences the fraction surviving, that is, with any given treatment, the higher the fraction of hematopoietic cells that are proliferating, the greater fraction of the total number is killed. Conversely, the greater the fraction of hematopoietic cells that are resting, that is, that are mitotically quiescent, the greater the fraction of cells that survive the toxic effects of chemotherapy

Normally, the fraction of a given population of cells, which are proliferating, is regulated by the balance of inhibiting and stimulating factors. If the proliferation of cells is increased by an exogenous factor, the absolute number of cells produced will be higher. This higher number will result in the decrease in endogenous stimulating factors and an increase in endogenous inhibiting factors.

If exogenous stimulation is stopped, this results in relative inhibition of proliferative activity with many cells entering a quiescent period or a period of “mitotic quiescence.”

Hematopoietic cell stimulating factors (“HCSFs”) are those substances, which stimulate the growth, proliferation and cellular output of hematopoietic cells. HCSFs include “cytokines” and “hematopoietic growth factors,” two broad classes of soluble protein hormones which are, in general, produced by various cells and have effects on the growth, differentiation, and function of other cells. Many of these proteins have been identified, purified, and their amino acid sequences have been determined. Their genes have also been identified, and cloned into recombinant genetic sequences of bacteria, yeast, and mammalian cells, which have been applied to the pharmaceutical production of these hormones. The methods of the present invention use HCSFs to induce cellular mitotic quiescence prior to and during periods of chemotherapy. By inducing such quiescence, a significant increase is achieved in the absolute number of hematopoietic cells, which survive chemotherapy.

A large number of HCSFs and cytokines, have been described and produced. Examples include Colony Stimulating Factors (“CSFs”), Interleukins, Interferons, Tumor Necrosis Factor, Erythropoietin, Thrombopoietin, and various so-called fusion proteins produced by biological engineering methods, which contain functional domains from various cytokine-type HCSFs that occur naturally. The availability of pure proteins has accelerated the understanding of their effects in experimental systems, and has also made them available for clinical testing, and eventual approved use. The CSFs Granulocyte-Macrophage Colony Stimulating Factor (GM-CSF, (Leukine®)) and Granulocyte Colony Stimulating Factor (G-CSF, Neupogen®)) are two such cytokines now in use for various indications, including cancer-associated neutropenia.

Chemotherapeutic dynamics and regimens are described in many standard reference texts used by practitioners of oncology. These texts include, for example, “Cancer: Principles and Practice of Oncology,” edited by DeVita, Hellman, and Rosenberg (Lippincott-Raven, 1997). As one of skill in the art will appreciate, the DeVita text, and other texts in the art, draw on many references in the relevant biomedical literature in detailing methods for effecting chemotherapies.

Methods or substances which prevent damage such as that caused by chemotherapy, or that promote the recovery of already damaged cell populations, are thus of considerable interest because such methods serve to preserve the health of the patient and allow a maximally aggressive chemotherapeutic attack on the cancer.

With the goal of ameliorating the myelosuppression associated with chemotherapy, a number of conventional investigations and methods have made use of CSFs and other cytokines in order to support recovery of hematopoietic cells following chemotherapy. More recent methods are illustrated, for example, by U.S. Pat. No. 5,496,804 to Reed and U.S. Pat. No. 5,595,973 to Bogden. The method of Bogden includes the administration of hematopoietic inhibitory factors during chemotherapy. Then, chemotherapy is followed by treatment with cytokines with the goal of stimulating the surviving hematopoietic cells (“the product cells”), to accelerate their recovery from the insult of the chemotherapy. The method of Reed includes the administration of granulocyte colony stimulating factor during a taxol regimen in order to counter the side effects of the taxol.

In contrast, the method of the present invention involves combining chemotherapy and the administration of one or more HCSFs in a sequence and regimen previously undescribed. Specifically, the present method effects the stimulation of hematopoietic cells by one or more HCSFs prior to the administration of chemotherapeutic agents. The stimulation of hematopoietic cells in accordance with the present method results in an increased population of hematopoietic cells both before and after chemotherapeutic treatment. In one aspect of the present invention, the method comprises stimulating the activities and proliferation of hematopoietic cells by administering to them sufficient quantities of HCSFs, and for a sufficient period of time, so that their population increases over a baseline or normal level, and preferably to the highest level achievable without unacceptable side effects.

When the size and activities of the hematopoietic cell population reach a desired level, administration of HCSFs is decreased, or preferably ceased, to provide a post-stimulation period in which proliferative activity ceases or diminishes greatly. During this post-stimulation period, or period of post-mitotic quiescence, proliferative activity diminishes greatly in response to both 1) the decrease of exogenous stimulating factors, 2) the decrease of endogenous stimulating factors, and 3) the increase in endogenous inhibitory substances released by feedback mechanisms in the hematopoietic cell population. Both 2) and 3) are caused by the higher than baseline population of hematopoietic cells induced by the administration of HCSFs.

One of the desired effects of inducing this period of intense proliferative activity by stimulation with HCSFs, followed by a post-stimulation period of no or little exogeneous HCSFs, is that the population of hematopoietic cells enters a period of post-mitotic quiescence. In turn, this proliferative activity produces an increase in the population of cells and thus an increase in the absolute number of cells surviving the chemotherapy.

Thus, according to the present invention, treatment with HCSFs precedes chemotherapy. Thus, the efficacy of the present method flows both from the population increase caused by the proliferative stimulation provided by the one or more HCSFs administered, and from the post-stimulation period of mitotic quiescence that follows a period of intense proliferative activity.

### SUMMARY OF THE INVENTION

It is therefore an object of the present invention to provide methods for minimizing the toxic effects which chemotherapies, radiation therapies and related therapies have on hematopoietic and other mitotically active cells of a patient.

It is a further object of the present invention to provide methods that can efficaciously be used with known as well as yet unknown anti-neoplastic and anti-cancer therapeutic chemical agents.

In accordance with these and other objects of the invention, a method for minimizing the toxic effects of chemotherapeutic agents or cytotoxic irradiation on the hematopoietic cells of a patient having neoplastic cells or a malignant tumor is provided. In accordance with one aspect of the invention the method comprises the steps of: A, treating the patient with a dosage of at least one hematopoietic cell stimulating factor, the dosage being sufficient in amount and time to cause a substantial increase in the population of the hematopoietic cells and in differentiated blood cells, and B, treating the patient with a dosage of chemotherapeutic agents or cytotoxic irradiation sufficient to substantially reduce the population of neoplastic cells.

In accordance with other aspects of the invention, the relative timing of Steps A and B can be varied in order to maximize the efficacy of the method. Thus, variations of methods according to the invention can be tailored to individual patients. For example, Step B can be initiated immediately after the cessation of Step A, or Step B can be delayed for a period of time, whichever is effective to ensure that the patient's population of hematopoietic cells is at a maximum level. Alternatively, Step B can be initiated before the cessation of Step A if necessary to maximize a patient's population of hematopoietic cells and minimize the destructive effects of chemotherapy or radiation therapy.

Thus, the methods of the invention provide further that, after Step A and before Step B, Step C provides for allowing a sufficient amount of time to elapse so that the hematopoietic cells enter a state of post-mitotic quiescence before being subjected to chemotherapeutic agents or irradiation. Quiescent cells are not dividing and therefore are not subject to the damaging effects of chemotherapeutic agents or irradiation. By providing Step C, the methods of the invention further shield the extant cells from damage.

In accordance with other aspects of the invention, Step A is performed for a sufficient period of time to stimulate the hematopoietic cells to enter a phase of proliferative activity sufficient to produce a crest in the population of the hematopoietic cells. By “crest” is meant a substantial increase or peak in the population of hematopoietic cells, and preferably an increase in the absolute number of cells to the maximum extent possible using hematopoietic cell stimulating factors (“HCSF's”). An additional advantage of this aspect of the invention is that an increase or peak in the population of hematopoietic cells produces a corresponding increase in the population of those cells, which differentiate from hematopoietic cells. Thus, many more differentiated blood cells are caused to be in circulation and are thus available to provide additional immune protection for a patient undergoing treatment for cancer.

The methods of the present invention can be performed with any medically-suited compound or compounds, which stimulate an increase in the population of hematopoietic cells. Preferably, the at least one hematopoietic cell stimulating factor is a cytokine, hematopoietic growth factor, fusion protein having functional domains of any of the cytokines or hematopoietic growth factors, or an agonist of any of the cytokines or hematopoietic growth factors. The cytokine or hematopoietic growth factor can be one or more selected from the group consisting of Colony Stimulating Factors, Interleukins, Interferons, Tumor Necrosis Factor, Erythropoietin, and Thrombopoietin,

In accordance with the methods of the invention, the Colony Stimulating Factors (“CSF's”) are preferably one or more from the group consisting of Granulocyte Macrophage Colony Stimulating Factor (“GMCSF”), Macrophage Colony Stimulating Factor (“MCSF”) and Granulocyte Colony Stimulating Factor “(GCSF”). Interleukins suitable for practicing the invention include one or more of IL-1, IL-2, IL-3, IL-6, IL-6, IL-11 and IL-12.

In accordance with the present invention, the methods may be used to treat many neoplasias and malignant tumors. Examples of such include lymphomas, Hodgkins disease, Wilm's tumor, embryonal rhabdomyosarcoma, small cell lung cancer, central nervous system lymphoma, anal carcinoma, bladder carcinoma, breast cancer, laryngeal cancer, osteogenic sarcoma, soft tissue sarcomas, nonsmall cell lung cancer, breast cancer, nasopharyngeal cancer, other cancers of the head and neck region, pancreatic cancer, gastric carcinoma, prostate cancer, and cervical carcinoma.

In accordance with additional objects, the methods according to the invention are applicable for minimizing the toxic effects of many chemotherapeutic drugs. Examples of such drugs include alkylating agents which interfere with the nucleic acid or protein metabolism of dividing cells such as Cyclophosphamide, Busalfan, Ifosfamide, Procarbazine, Dacarbazine, Temozolomide, Hexamethylmelamine, and ThioTEPA.; topoisomerase inhibitors such as Etoposide, Doxo-rubicin Epirubicin and Amonafide., drugs that interfere with DNA synthesis such as Cytosine Arabnoside, Mercaptopurine, methotrexate, fluorouracil, Cisplatin, Carboplatin, Oxaliplatin, JM216, CI-973, DWA 2114R, JM335, Bisplatinum and analogues thereof; drugs that interfere with the synthesis and assembly of microtubuies or the mitotic spindle apparatus such as Vincristine and Paclitaxel; and antibiotics such as Mitomycin C and Actinomysin D.

### DETAILED DESCRIPTION OF THE INVENTION

TABLE 1
The Korkut method (CSF-treatment prior to chemotherapy) vs.
the current standard method of chemotherapy followed by CSF-rescue

| Variables: | Current Method: | Korkut Method: |
|---|---|---|
| in therapy order, | Chemotherapy followed by | CSF treatment prior to |
| physiological context, | CSF rescue treatment | chemotherapy |
| and effects ... | | |
| Relative Order of Chemo- | chemotherapy is followed by | CSF-pretreatment is followed by |
| and CSF-Therapies: | CSF-rescue therapy | chemotherapy |
| Chemotherapy: | | |
| Status of progenitor cell | a normal baseline cell population | a CSF treatment-increased cell |
| population upon initiation | level, proliferating normally | population level, proliferating |
| of chemotherapy | | slowly (due to post-proliferative- |
| | | burst quiescence) |
| Response of progenitor cell | the cell population is damaged | the cell population is spared |
| population to chemotherapy | by chemotherapy approximately | damage in proportion to the |
| | in proportion to its proliferative | decrease in proliferative rate; |
| | rate during treatment | also a larger population yields |
| | | an increased absolute level of |
| | | surviving hematopoietic cells |
| CSF-Therapy: | | |
| Status of progenitor cell | a chemo-surviving population at | a normal baseline cell |
| population upon initiation | a below-normal level | population, with a normal |
| of CSF therapy | | proliferative rate, and normal |
| | | capacity to respond to |
| | | proliferative stimulation |
| Response of progenitor cell | the diminished population | the normal population, prolifer- |
| population to CSF therapy | responds in a less than optimal | ating normally, responds to |
| | way to CSF-proliferative stimuli | CSF-treatment optimally by |
| | | increasing proliferation to an |
| | | above-normal rate |
| Post-Chemotherapy: | | |
| Post-treatment status of | a decreased population of progen- | a relatively high population of |
| progenitor cell and WBC | itor cells, responding poorly to | progenitor cells, responding to |
| populations | CSF-proliferative stimuli; | CSF-proliferative stimuli; |
| | and a peripheral WBC level well | and a peripheral WBC level |
| | below normal | closer to normal than in the case |
| | | of conventional chemotherapy |

The methods of the present invention are directed toward effecting the protection of hematopoietic cells from the cytotoxic effects of therapeutic irradiation or cancer chemotherapy in which patients are treated with HCSFs, such as cytokines or hematopoietic growth factors. An important aspect of the present methods is that administration of HCSFs is begun prior to administration of toxic treatment such as chemotherapy. By practicing the present methods, hematopoietic cells are substantially protected from cytotoxic damage which would otherwise result from the chemotherapy.

More specifically, the method of this invention includes the steps of (1) administering to a patient a dosage of one or more HCSFs effective in promoting the proliferation of hematopoietic progenitor cells, and (2) at an appropriate time near the end of the administration of the HCSFs, or after cessation of HCSF treatment, administering chemotherapy to the patient.

Examples of HCSFs which can be used to practice this invention include, but are not limited to: Colony Stimulating Factors (such as GM-CSF, G-CSF, and M-CSF), Interleukins, Interferons, Tumor Necrosis Factor, Erythropoietin, and Thrombopoietin. In addition to the naturally occurring HCSFs, this invention can also be practiced with biologically engineered modified versions of these substances, as well as various so-called fusion proteins which contain functional domains from different naturally occurring HCSFs.

In accordance with the present invention, optimum doses of HCSFs are administered for a particular patient in amounts appropriate to maximize the increase in the population of hematopoietic cells and to induce a period of post-mitotic quiescence during which chemotherapy can be administered. Actual dosages depend upon the response of the specific patient to the administration of the HCSFs.

By chemotherapy is meant treatment of a patient with various cytotoxic agents which kill proliferating cells. Examples of such chemotherapeutic drugs include, but are not limited to, cyclophosphamide, taxol, 5-fluorouracil, Adriamycin, cisplatinum, methotrexate, cytose arabinoside, mitomysin C, vindesine, carboplatinum, vincristine, and agonists and modified versions thereof. As one of skill in the art will recognize, the typical period of administration of chemotherapeutic agents ranges from approximately 1-5 days, depending on the type of cancer being treated, and other factors specific to the particular patient.

With reference to FIG. 1 and Table 1, the present invention can be understood against the contrasting background of the prior art.

FIG. 1 compares the time course of treatment by (a) a standard prior art form of chemotherapy without any conjunctive treatment with HCSFs, (b) a standard prior art form of chemotherapy which is then followed by cytokine therapy, and (c) the present invention, in which HCSF therapy, and a post-stimulation period, precedes chemotherapy. The chart and graphs depict the number of the hematopoietic progenitor cells, the relative increase or decrease in the number of these cells before, during and after treatment, and the levels of circulating white blood cells under these three regimens. Table 1 is a more detailed comparison of the latter two forms of therapy, the standard prior art form in which HCSF treatment follows chemotherapy and the present invention in which HCSF treatment precedes chemotherapy. The Table focuses on the status of the progenitor cells, their response to treatments under the two regimens, and the post-therapy status of progenitor cells and circulating white blood cells.

There are at least two key ways in which the patient status following therapy is improved by the present invention over methods of the prior art. One way is that levels of both progenitor cell populations and circulating white blood cells are higher than before the administration of chemotherapy. A second way is that the increase in the absolute number of cells in the hematopoietic progenitor cell population provides a higher absolute number of cells which survive the administration of chemotherapy. Moreover, as an effect of pre-chemotherapy administration of one or more HCSFs, the higher number of circulating white blood cells, by releasing naturally occurring inhibitory factors, increases the inhibitory effects on the progenitor cell population thereby decreasing their mitotic activity to a level of post-mitotic quiescence thereby protecting them from the cytotoxic effects of chemotherapeutic agents.

### BRIEF DESCRIPTION OF THE DRAWINGS

FIG. 1. is a three-part composite schematic which compares patient numbers of hematopoietic progenitor cells, the increase or decrease in the number of hematopoietic progenitor cells, and white blood cell levels over the time course of three types of therapy: (a) conventional chemotherapy, (b) prior art form of chemotherapy followed by CSF rescue therapy, and (c) the present invention method of pretreatment with HCSF to induce mitotic quiescence prior to chemotherapy.

Table 1 is a detailed comparison of (a) the prior art form of chemotherapy followed by CSF rescue therapy, and (b) the presently-described new method of CSF-pretreatment prior to chemotherapy with respect to the physiological context and response of hematopoietic progenitor cells during the chemotherapy and HCSF-treatment portions of these two methods respectively.

### DESCRIPTION OF THE PREFERRED EMBODIMENTS

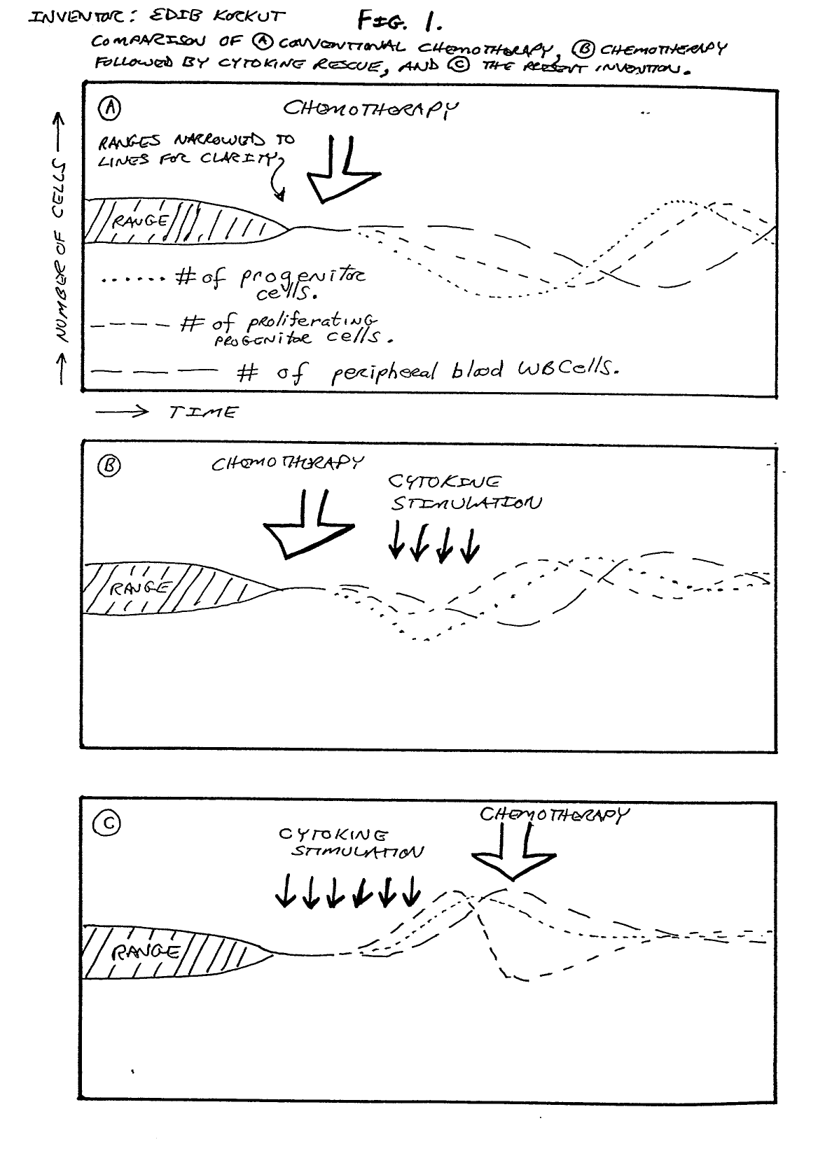

FIG. 1, panel C depicts a preferred embodiment of therapy by the method this invention describes. This is a representation of a single cycle of HCSF therapy followed by chemotherapy. Such cycles can be repeated two, three, or more times over a full course of cancer therapy, if necessary. The present invention is embodied within the context of a single cycle, regardless of how many cycles are conducted during the total course of therapy. That is, as one of skill in the oncological arts will comprehend, repetition of the cycle of the present invention will sometimes be necessary and sometimes be unnecessary in order to maximize its benefit to a particular patient.

The initial phase of treatment in accordance with the present invention involves administration of Hematopoietic Cell Stimulating Factors, such as one or more cytokines or hematopoietic growth factors such as GM-CSF or G-CSF, in an amount and for a period of time sufficient to increase significantly the number of hematopoietic progenitor cells and to induce post-mitotic quiescence. Other embodiments make use of one or more of the other soluble protein cytokines or hematopoietic growth factors, including but not limited to, Interleukins, Interferons, Tumor Necrosis Factor, Erythropoietin, and Thrombopoietin, as well as biologically-engineered and modified versions thereof, including fusion proteins which contain functional domains from different naturally occurring cytokines.

During the period of treatment with one or more HCSFs, there is an increase in the rate of proliferation among the hematopoietic progenitor cell populations, which results in higher base levels of these cells. During this period, also, there is a resulting increase in the level of circulating peripheral white blood cells.

Following the initial phase of treatment with one or more HCSFs, there follows a period during which the exogenous stimulation provided by the administered HCSFs is removed. As a result of this lack of exogenous stimulation, and as a result of counteracting inhibiting factors induced by a higher than normal number of circulating white blood cells, the proliferative rate of the progenitor cells falls significantly below normal for a time sufficient for the administration of chemotherapeutic agents.

According to the methods of the present invention, chemotherapy is thus initiated after the cell stimulation phase and during the post-stimulation period of post-mitotic quiescence. Of course, as described above, the relative timing of the steps of the present methods may be varied when necessary to maximize its effectiveness. Thus, a particular regimen of treatment may require that chemotherapy or radiation therapy commence before the cessation of HCSF administration.

As one of skill will appreciate, the present invention, however, does not have an embodiment which is preferred over any other with respect to the specifics of the chemotherapeutic drug, dosage, duration of treatment, or number of cycles of such treatment, because such specifics follow from the particular form of cancer being treated, the response of the individual patient to treatment and the availability of particular HCSFs. The present invention applies generally to all forms of cancer that are treatable with chemotherapy or radiation, with the possible exception of leukemias.

Examples of chemotherapeutic drugs useful in practicing the present invention include, but are not limited to, cyclophosphamide, taxol, 5-fluorouracil, adriamycin, cisplatinum, methotrexate, cytose arabinoside, mitomysin C, vindesine, carboplatinum, vincristine, and agonists and modified versions thereof.

The foregoing description has been limited to specific embodiments of the methods of the present invention. It is clear, however, that variations and modifications may by made to the present invention, which would be within the scope and spirit of the invention, and still yield some or all of its advantages.

What is claimed is:

1. A method for minimizing the toxic effects of chemotherapeutic agents or cytotoxic irradiation on the hematopoietic cells of a patient having neoplastic cells or a malignant tumor, comprising the steps of:
A. treating the patient with a dosage of at least one hematopoietic cell stimulating factor, the dosage being sufficient in amount and time to cause a substantial increase in the population of the hematopoietic cells and in differentiated blood cells, and
B. treating the patient with a dosage of chemotherapeutic agents or cytotoxic irradiation sufficient to substantially reduce the population of neoplastic cells.

2. The method of claim 1, wherein Step B is initiated after the cessation of Step A.

3. The method of claim 1, wherein Step B is initiated before the cessation of Step A.

4. The method of claim 1, wherein after Step A and before Step B, further comprising Step C, allowing a sufficient amount of time to elapse so that the hematopoietic cells enter a state of post-mitotic quiescence.

5. The method of claim 1, wherein Step A is performed for a sufficient period of time to stimulate the hematopoietic cells to enter a phase of proliferative activity sufficient to produce a crest in the population of the hematopoietic cells.

6. The method of claim 1, wherein the at least one hematopoietic cell stimulating factor is a cytokine, hematopoietic growth factor, fusion protein having functional domains of any of the cytokines or hematopoietic growth factors, or an agonist of any of the cytokines or hematopoietic growth factors.

7. The method of claim 6, wherein the cytokine or hematopoietic growth factor is one or more selected from the group consisting of Colony Stimulating Factors, Interleukins, Interferons, Tumor Necrosis Factor, Erythropoietin, and Thrombopoietin,

8. The method of claim 7 wherein the Colony Stimulating Factors are one or more from the group consisting of Granulocyte Macrophage Colony Stimulating Factor, Macrophage Colony Stimulating Factor and Granulocyte Colony Stimulating Factor.

9. The method of claim 7 wherein the Interleukins are one or more selected from the group consisting of IL-1, IL-2, IL-3, IL-6, IL-6, IL-11 and IL-12.

10. The method of claim 1, wherein Step B is performed while the hematopoietic cells are in a state of post-mitotic quiescence.

11. The method of claim 1, used to treat one or more cancers from the group consisting of lymphomas, Hodgkins disease, Wilm's tumor, embryonal rhabdomyosarcoma, small cell lung cancer, central nervous system lymphoma, anal carcinoma, bladder carcinoma, breast cancer, laryngeal cancer, osteogenic sarcoma, soft tissue sarcomas, nonsmall cell lung cancer, breast cancer, nasopharyngeal cancer, other cancers of the head and neck region, pancreatic cancer, gastric carcinoma, prostate cancer, and cervical carcinoma.

12. The method of claim 1, wherein the chemotherapeutic drugs are one or more substances selected from the following groups: alkylating agents which interfere with the nucleic acid or protein metabolism of dividing cells, topoisomerase inhibitors, drugs that interfere with DNA synthesis, drugs that interfere with synthesis and assembly of microtubules or the mitotic spindle apparatus, and antibiotics.

13. The method of claim 10, wherein the alkylating agents are one or more selected from the group consisting of Cyclophosphamide, Busalfan, Ifosfamide, Procarbazine, Dacarbazine, Temozolomide, Hexamethylmelamine, and ThioTEPA.

14. The method of claim 10, wherein the topoisomerase inhibitors are one or more from the group consisting of Etoposide, Doxo-rubicin Epirubicin and, Amonafide.

15. The method of claim 10, wherein the antibiotics are one or more selected from the group consisting of Mitomycin C, and Actinomysin D.

16. The method of claim 10, wherein the drugs that interfere with DNA synthesis are one or more selected from the group consisting of Cytosine Arabnoside, Mercaptopurine, Methotrexate, Fluorouracil, Carboplatin, Oxaliplatin, JM216, CI-973, DWA 2114R, JM335, Bisplatinum and analogues thereof.

17. The method of claim 10, wherein the drugs that interfere with synthesis and assembly of microtubules and the mitotic spindle apparatus, are one or more selected from the group consisting of Vincristine and Paclitaxel.
